(19) United States
(12) Patent Application Publication
(10) Pub. No.: US 2018/0177636 A1
(43) Pub. Date: Jun. 28, 2018

(54) SIDE FENESTRATED PARS PLANA INFUSION CANNULA

(71) Applicant: Harry Michael Lambert, Austin, TX (US)

(72) Inventor: Harry Michael Lambert, Austin, TX (US)

(21) Appl. No.: 15/834,379

(22) Filed: Dec. 7, 2017

Related U.S. Application Data
(60) Provisional application No. 62/439,404, filed on Dec. 27, 2016.

(51) Int. Cl. A61F 9/007

(52) U.S. Cl. CPC A61F 9/00736; A61B 17/3421

(57) ABSTRACT

A cannula designed to be used during vitreoretinal and ocular surgery may include an intraocular portion with a tip; and at least two fenestrations extending through the intraocular portion proximate to the tip. The fenestrations may be angled so as to avoid or reduce perpendicular injection and maximize posterior injection.

### RELATED APPLICATION

This application claims priority to provisional patent application U.S. Ser. No. 62/439,404 filed on Dec. 27, 2017, the entire contents of which is herein incorporated by reference.

### BACKGROUND

The embodiments herein relate generally to medical devices, and more particularly, to side fenestrated pars plana infusion cannulas for use in vitreoretinal and ocular surgery.

In vitreoretinal and ocular surgery, existing infusion cannulas infuse from the end of the cannula. As a result of this structure, the current cannulas create a “jet stream” of fluid or gas being injected directly into the eye, which can hit the opposite side of the retina and potentially damage it. To address this issue, some cannulas include a diverter to divert at least some of the fluid exiting the cannula. However this device still injects straight into the eye.

Therefore, what is needed is a cannula designed to lessen the pressure of the infusion at a given infusion site to prevent the “jet stream” effect associated with existing cannulas during vitreoretinal and ocular surgery, while injecting toward the central bulk of the eye and not into the periphery.

### SUMMARY

Some embodiments of the present disclosure include a cannula designed to be used during vitreoretinal and ocular surgery. The cannula may include an intraocular portion with a tip; and at least two fenestrations extending through the intraocular portion proximate to the tip. The fenestrations may be angled so as to avoid or reduce perpendicular injection and maximize posterior injection.

### BRIEF DESCRIPTION OF THE FIGURES

The detailed description of some embodiments of the invention is made below with reference to the accompanying figures, wherein like numerals represent corresponding parts of the figures.

FIG. 1 is a side elevation view of one embodiment of the present disclosure.

FIG. 2 is an enlarged side elevation view of one embodiment of the present disclosure.

FIG. 3 is a cross-sectional view of one embodiment of the present disclosure, taken along line 3-3 in FIG. 1.

FIG. 4 is a side elevation view of one embodiment of the present disclosure.

FIG. 5 is a side elevation view of one embodiment of the present disclosure.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used as a cannula for vitreoretinal and ocular surgery and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

a. Cannula

b. Multiple Fenestrations

The various elements of the device of the present disclosure may be related in the following exemplary fashion. It is not intended to limit the scope or nature of the relationships between the various elements and the following examples are presented as illustrative examples only.

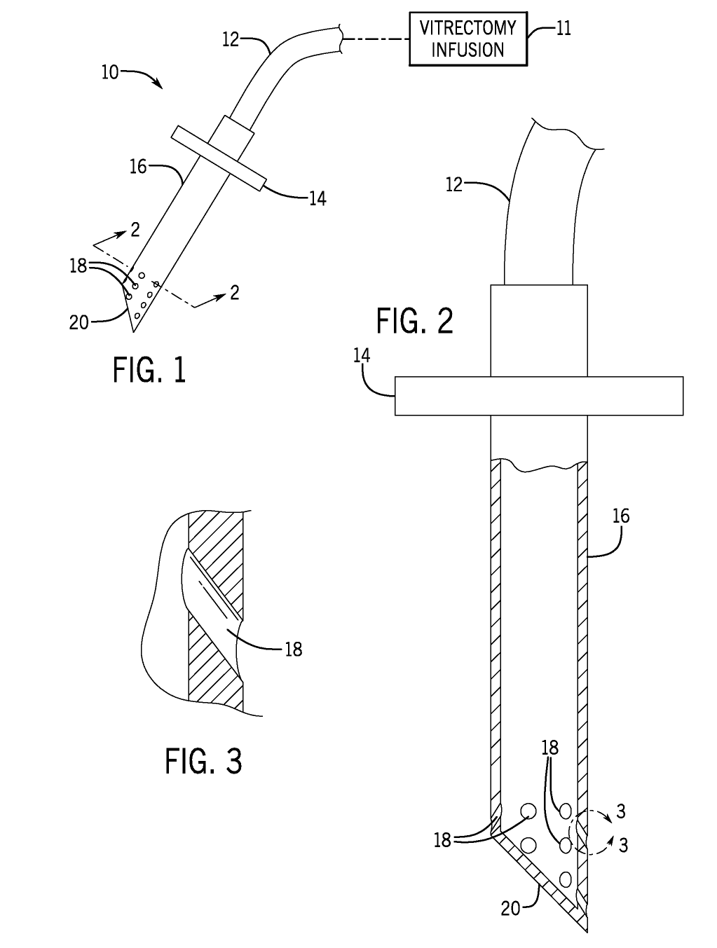

By way of example, and referring to FIGS. 1-13, some embodiments of the present disclosure include a cannula designed to be used during vitreoretinal and ocular surgery, the cannula comprising an intraocular portion 16 comprising a plurality of fenestrations 18 extending therethrough. Specifically, the cannula may comprise an intraocular portion 16 extending from a guard 14, wherein the intraocular portion 16 comprises a tip, and the plurality of fenestrations 18 may start proximate to the tip and be spaced a long a length of the intraocular portion 16 toward the guard 14. In embodiments, the fenestrations 18 may comprise side fenestrations and may be angled to allow for optimal safe injection of fluids during the surgery. While the figures show a large number of fenestrations 18, embodiments of the cannula may include as few as two fenestrations 18 extending through the intraocular portion 16. However, the increase in the number of fenestrations 18 may result in less pressure and, therefore, less hitting of the other side of the retina. The cannula may further comprise infusion tubing 12 extending from an end of the cannula distal from the tip.

A first example of a cannula of the present disclosure is shown in FIGS. 1 and 2. As shown in these figures, the cannula 10 may be designed to work with current systems, wherein an end of the cannula 10 distal from the fenestrations 18 may be compatible with conventional systems and attached to a conventional vitrectomy device 11. The intraocular portion 16 may comprise an angled tip 20 and a plurality of fenestrations 18 extending through and positioned proximate to the pointed tip 22, wherein the fenestrations 18 may be angled, as shown in FIG. 3, to avoid or reduce perpendicular injection and maximize posterior injection.

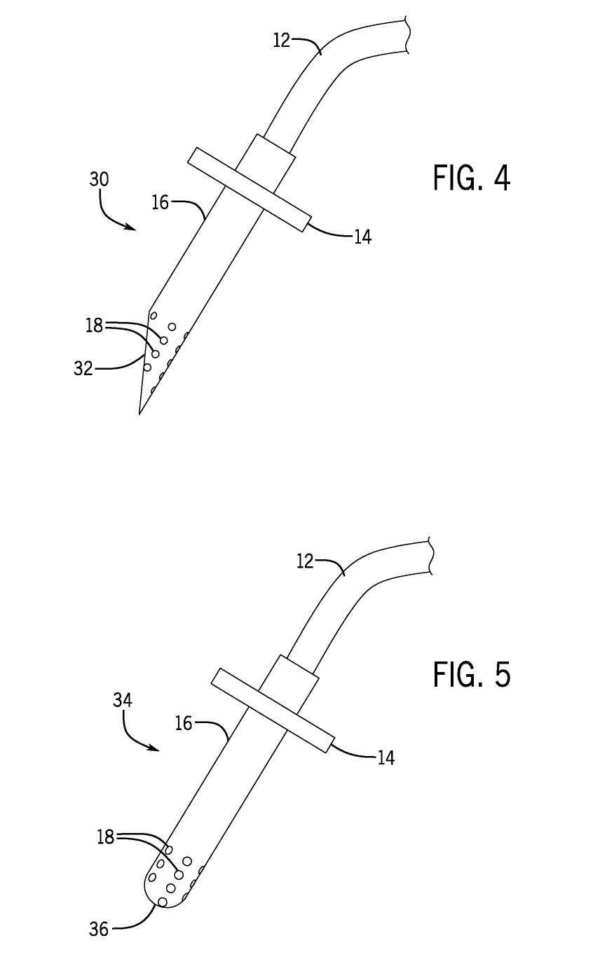

A second example of a cannula of the present disclosure is shown in FIG. 4. As shown in FIG. 4, the cannula may be a self-incising cannula 30, wherein the tip is a sharp, self-incising tip 32, such that a separate sclerotomy is not required. The sharp, self-incising tip 32 may comprise a plurality of fenestrations 18 positioned proximate thereto, wherein the fenestrations 18 may be angled, as shown in FIG. 3, to reduce perpendicular injection and maximize posterior injection.

A third example of a cannula of the present disclosure is shown in FIG. 5. As shown in FIG. 5, the cannula may be a blunt cannula 34, wherein the tip is a rounded tip 36. The rounded tip 36 may comprise a plurality of fenestrations 18 positioned proximate thereto, wherein the fenestrations 16 may be angled, as shown in FIG. 3, to avoid or reduce perpendicular injection and maximize posterior injection.

In any of the above embodiments, an external portion of the cannula (i.e., the end of the cannula distal from the intraocular portion), may be sutured, self-stabilizing, or mesh with the cannula.

In the case of the cannula 10 and the blunt cannula 34, the cannulas may be designed to fit through a separate cannula for insertion. Alternatively, the self-incising cannula 30 may allow for self-insertion and, thus, may not require the separate cannula for insertion. In other words, the self-incising cannula 30 may itself be the cutting device, negating the need for a separate cutting device.

The cannula of the present disclosure may have any necessary size and shape to match the vitrectomy system 11 being used, such that it functions in concert with the system. The cannula may also be made of any suitable materials. Lastly, the fenestrations may have any desired diameter and, in some embodiments, the total area of the fenestrations may be approximately equal to the orifice in a standard cannula.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A cannula designed to be used during vitreoretinal and ocular surgery, the cannula comprising:
an intraocular portion with a tip; and
at least two fenestrations extending through the intraocular portion proximate to the tip.

2. The cannula of claim 1, wherein the at least two fenestrations are angled through the intraocular portion.

3. The cannula of claim 1, wherein the tip is selected from the group consisting of an angled tip, a self-incising tip, and a blunt tip.

4. The cannula of claim 1, further comprising a guard positioned proximate to an end of the cannula distal from the tip.

5. The cannula of claim 4, wherein the at least two fenestrations start proximate to the tip and are spaced a long a length of the intraocular portion toward the guard.

6. The cannula of claim 1, further comprising infusion tubing extending from an end of the cannula distal from the tip.
